(19) United States
(12) Patent Application Publication
(10) Pub. No.: US 2023/0005612 A1
(43) Pub. Date: Jan. 5, 2023

(54) PHYSICAL CONDITION DETECTION METHOD, PHYSICAL CONDITION DETECTION DEVICE, AND RECORDING MEDIUM

(71) Applicant: Panasonic Intellectual Property Corporation of America, Torrance, CA (US)

(72) Inventor: Kenji MASUDA, Osaka (JP)

(21) Appl. No.: 17/943,469

(22) Filed: Sep. 13, 2022

Related U.S. Application Data
(63) Continuation of application No. PCT/JP2021/011463, filed on Mar. 19, 2021.

(30) Foreign Application Priority Data
Mar. 30, 2020 (JP) 2020-059718

(51) Int. Cl. G16H 40/63

(52) U.S. Cl. CPC G16H 40/63

(57) ABSTRACT

A physical condition detection method executed by a computer includes: obtaining body motion data of a subject over a given current time period including a current time; generating a current state model representing a current state of physical condition of the subject from the body motion data of the subject over the current time period, by using a mathematical model constructed using body motion data of the subject over a past period during which the physical condition of the subject was in a normal state; and outputting the current state model for detecting a change in the physical condition of the subject based on one or more differences obtained by comparing the current state model and a normal state model representing the normal state of the physical condition of the subject and generated from the body motion data of the subject over the past period using the mathematical model.

### CROSS REFERENCE TO RELATED APPLICATIONS

This is a continuation application of PCT International Application No. PCT/JP2021/011463 filed on Mar. 19, 2021, designating the United States of America, which is based on and claims priority of Japanese Patent Application No, 2020-059718 filed on Mar. 30, 2020. The entire disclosures of the above-identified applications, including the specifications, drawings and claims are incorporated herein by reference in their entirety.

### FIELD

The present disclosure relates to a physical condition detection method, a physical condition detection device, and a recording medium.

### BACKGROUND

For example, Patent Literature (PTL) 1 has proposed a system for attending someone's deathbed that allows a close relative in a remote location to be present at the death of a closely related person. PTL 1 discloses obtaining an estimated time of death of a subject from a current time, based on information indicating correlation between (i) information about each change, such as a change in electrocardiograms (ECGs), heart rates, and so on obtained from many past death cases and (ii) information about how long each person lived after such a change.

### CITATION LIST

### Patent Literature

PTL 1: Japanese Unexamined Patent Application Publication No. 2017-33502

### SUMMARY

### Technical Problem

However, it is difficult to gather a large amount of data about past death cases. Even if a large amount of data of past death cases could be gathered, changes in ECGs and heart rates greatly vary from person to person. Therefore, an estimated time from the current time to the time of death that can be obtained based on the information indicating the above correlation is expected to be approximately several hours.

On the other hand, it is relatively easy to gather data (referred to as body motion data), such as heart rates, respiration, and body motion, on the closely related person, i.e., the subject. Moreover, it is also desired to detect a change in the physical condition of the subject early, including a change in physical condition toward death during terminal care.

The present disclosure has been conceived in view of the above situations, and provides a physical condition detection method, a physical condition detection device, and a recording medium capable of detecting a change in physical condition of a subject.

### Solution to Problem

In order to achieve the above, a physical condition detection method according to one aspect of the present disclosure is a physical condition detection method executed by a computer, The physical condition detection method includes: obtaining body motion data of a subject over a current time period that is a given time period including a current time; generating a current state model representing a current state of physical condition of the subject from the body motion data of the subject over the current time period, by using a mathematical model constructed using body motion data of the subject over a past period during which the physical condition of the subject was in a normal state; and outputting the current state model for detecting a change in the physical condition of the subject based on one or more differences obtained by comparing the current state model and a normal state model representing the normal state of the physical condition of the subject, the normal state model being generated from the body motion data of the subject over the past period using the mathematical model.

Note that, one or more specific aspects of the present disclosure may be implemented as a system, a method, an integrated circuit, a computer program, or a computer-readable recording medium such as a CD-ROM, or may be implemented as any combination of a system, a method, an integrated circuit, a computer program, and a recording medium.

### Advantageous Effects

The physical condition detection method, etc. according to the present disclosure makes it is possible to detect a change in physical condition of a subject.

### BRIEF DESCRIPTION OF DRAWINGS

These and other advantages and features will become apparent from the following description thereof taken in conjunction with the accompanying Drawings, by way of non-limiting examples of embodiments disclosed herein.

FIG. 1 is a diagram illustrating an example of a configuration of a physical condition detection system according to an embodiment.

FIG. 2 is a diagram illustrating an example of a configuration of a physical condition detection device according to the embodiment.

FIG. 3 is a diagram illustrating an example of a subject whose body motion data according to the embodiment is obtained.

FIG. 4A is a flowchart illustrating operation of the physical condition detection device according to the embodiment.

FIG. 4B is a flowchart illustrating operation of the physical condition detection device according to the embodiment.

FIG. 5A is a diagram illustrating a normal state model and a current state model according to Working Example 1 of the embodiment,

FIG. 5B is a diagram illustrating a normal state model and a current state model according to Working Example 1 of the embodiment,

FIG. 5C is a diagram illustrating a normal state model and a current state model according to Working Example 1 of the embodiment.

FIG. 6A is a diagram illustrating a normal state model and a current state model according to Working Example 2 of the embodiment,

FIG. 6B is a diagram illustrating a normal state model and a current state model according to Working Example 2 of the embodiment,

FIG. 6C is a diagram illustrating a normal state model and a current state model according to Working Example 2 of the embodiment.

FIG. 6D is a diagram illustrating a normal state model and a current state model according to Working Example 2 of the embodiment.

FIG. 6E is a diagram illustrating a normal state model and a current state model according to Working Example 2 of the embodiment,

FIG. 6F is a diagram illustrating a normal state model and a current state model according to Working Example 2 of the embodiment.

FIG. 6G is a diagram illustrating a normal state model and a current state model according to Working Example 2 of the embodiment.

FIG. 6H is a diagram illustrating a normal state model and a current state model according to Working Example 2 of the embodiment.

FIG. 6I is a diagram illustrating a normal state model and a current state model according to Working Example 2 of the embodiment.

FIG. 7 is a diagram illustrating an example of a configuration of a physical condition change detection device according to the embodiment,

FIG. 8 is a table showing a result of verification of performance when a mathematical model is constructed using features of seven patterns.

### DESCRIPTION OF EMBODIMENT

A physical condition detection method according to one aspect of the present disclosure is a physical condition detection method executed by a computer. The physical condition detection method includes: obtaining body motion data of a subject over a current time period that is a given time period including a current time; generating a current state model representing a current state of physical condition of the subject from the body motion data of the subject over the current time period, by using a mathematical model constructed using body motion data of the subject over a past period during which the physical condition of the subject was in a normal state; and outputting the current state model for detecting a change in the physical condition of the subject based on one or more differences obtained by comparing the current state model and a normal state model representing the normal state of the physical condition of the subject, the normal state model being generated from the body motion data of the subject over the past period using the mathematical model.

With this, a current state model for detecting a change in physical condition of a subject can be output using only the body motion data of the subject. In other words, a current state model that can detect a change in the physical condition of the subject can be output by comparing the normal state model and the current state model.

In this way, the physical condition detection method that can detect a change in the physical condition of the subject can be implemented.

Here, for example, the physical condition detection method further includes: obtaining the one or more differences by comparing the current state model and the normal state model; and notifying an alert based on the one or more differences.

With this, upon receipt of the alert, it is possible to know there has been a change in the physical condition of the subject.

Moreover, for example, the alert may be notified when a change in the physical condition of the subject is detected based on the one or more differences.

With this, upon receipt of the alert, it is possible to know a change in the physical condition of the subject has been detected.

Here, for example, the one or more differences are (i) a distance between the normal state model and the current state model and (ii) a direction of movement from a position where the normal state model is located to a position where the current state model is located, and the change in the physical condition of the subject is detected based on the distance and the direction.

Moreover, for example, before the generating of the current state model, generating the normal state model from the body motion data of the subject over the past period by using the mathematical model.

Moreover, for example, the physical condition detection method may further include: regenerating a normal state model representing the normal state of the subject from body motion data of the subject over a new past period including the current time period, when there is no difference between the normal state model and the current state model.

This makes it possible to use a time period that is closer to a current time and during which the physical condition of the subject was in a normal state to generate the normal state model, Therefore, this allows more accurate detection of a change in the physical condition of the subject.

Moreover, for example, the mathematical model may be constructed using features obtained by calculating statistics that are based on interaction, from the body motion data of the subject over the past period.

With this, a mathematical model that can generate a normal state model and a current state model can be constructed using only the body motion data of the subject.

A physical condition detection device according to one aspect of the present disclosure includes: an obtainer that obtains body motion data of a subject over a current time period that is a given time period including a current time; a processor that generates a current state model representing a current state of physical condition of the subject from the body motion data of the subject over the current time period, by using a mathematical model constructed using body motion data of the subject over a past period during which the physical condition of the subject was in a normal state; and an outputter that outputs the current state model for detecting a change in the physical condition of the subject based on one or more differences obtained by comparing the current state model and a normal state model representing the normal state of the physical condition of the subject, the normal state model being generated from the body motion data of the subject over the past period using the mathematical model.

Note that, one or more of specific aspects of the present disclosure may be implemented as a system, a method, an integrated circuit, a computer program, or a computer-readable recording medium such as a CD-ROM, or may be implemented as any combination of a system, a method, an integrated circuit, a computer program, and a recording medium.

Hereinafter, a physical condition detection method according to one aspect of the present disclosure will be specifically described with reference to the drawings. Note that the embodiment described below shows a specific example of the present disclosure. The numerical values, shapes, materials, structural elements, the arrangement and connection of the structural elements, etc. mentioned in the following embodiment are mere examples and not intended to limit the present disclosure. Of the structural elements in the following embodiment, structural elements not recited in any one of the independent claims representing broadest concepts are described as optional structural elements. In addition, the embodiments can be combined with one another.

### Embodiment

First, a physical condition detection system to be used for implementing a physical condition detection method will be described.

### [1. Physical Condition Detection System]

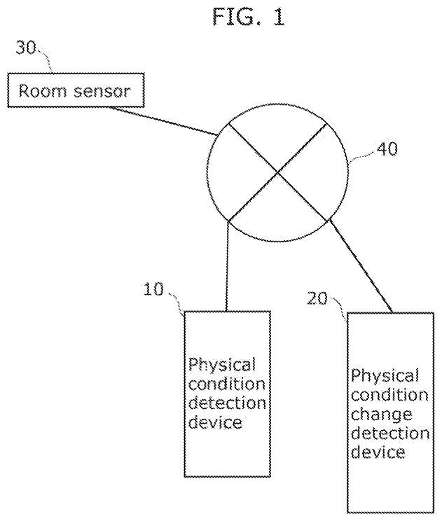

FIG. 1 is a diagram illustrating an example of a configuration of a physical condition detection system according to the present embodiment.

The physical condition detection system according to the present embodiment can detect a change in physical condition of a subject by including at least physical condition detection device 10. In the present embodiment, the physical condition detection system includes physical condition detection device 10, physical condition change detection device 20, and room sensor 30 as illustrated in FIG. 1. Physical condition detection device 10, physical condition change detection device 20, and room sensor 30 are connected by network 40.

Room sensor 30 is provided in a room where the subject is present, and obtains biological information of the subject. Physical condition detection device 10 obtains the biological information of the subject from room sensor 30, and generates and outputs information to be used for detecting the physical condition of the subject, Physical condition change detection device 20 uses the information output by physical condition detection device 10 to detect a change in the physical condition of the subject and notify the change. Note that room sensor 30 is an example, and may be any sensor capable of obtaining biological information of a subject. Room sensor 30 may be, for example, a sensor worn by the subject, or a seat-type sensor placed under a bed sheet or a mattress of a bed.

In the following, each device will be described.

### [2. Physical Condition Detection Device 10]

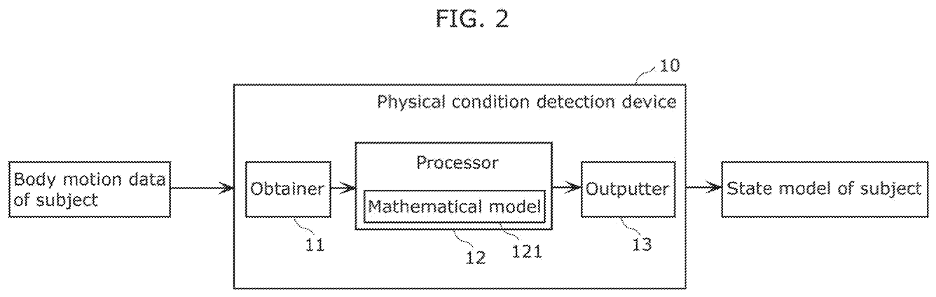

FIG. 2 is a diagram illustrating an example of a configuration of physic& condition detection device 10 according to the present embodiment.

Physical condition detection device 10 outputs a state mod& representing a state of physical condition of the subject. The state model is generated from the body motion data of the subject. In the present embodiment, physical condition detection device 10 includes obtainer 11, processor 12, and outputter 13 as illustrated in FIG. 2. Each structural element will be described in detail below.

### [2.1 Obtainer 11]

Obtainer 11 obtains body motion data of the subject from room sensor 30 via network 40, More specifically, obtainer 11 obtains body motion data of the subject in a current time period that is a given time period including a current time, Moreover, obtainer 11 may obtain body motion data of the subject over a past period.

Here, the body motion data includes, but is not limited to, heart rate information, respiratory information, and body motion information of the subject. The body motion data may include any other data of the biological information of the subject, The heart rate information includes the subject's time-series heart rate data associated with times of day, the respiratory information includes the subject's time-series respiratory data associated with times of day, and the body motion information includes the subject's time-series body motion data associated with times of day. Body motion is movement of the body. However, in medical practice, body motion is often referred to as unconscious movement of the body, such as movement during sleep.

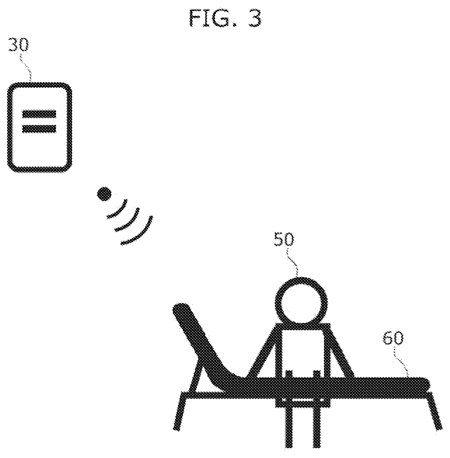

FIG. 3 is a diagram illustrating an example of the subject whose body motion data according to the embodiment is obtained.

Subject 50 illustrated in FIG. 3 is, for example, a terminal care patient at a hospice facility. The following three types of biological information of subject 50 are obtained by room sensor 30 placed in the hospice facility as body motion data: heart rate information, respiratory information, and body motion information. In the present embodiment, physical condition detection device 10 uses body motion data when subject 50 is sleeping in bed 60 and outputs information for detecting a change in the physical condition of the subject. Moreover, a change in physical condition in this case means that the physical condition of subject 50 changes toward death during terminal care.

Note that the subject whose physical condition is to be detected is not limited to a patient in a hospice facility mentioned in the above example. The subject may be a patient having myocardial infarction or apnea syndrome. Moreover, the subject whose physical condition is to be detected does not need to be a patient. The subject may be any person whose physical condition is desired to be detected using body motion data during sleep.

### [2.2 Processor 12]

Processor 12 uses mathematical model 121 to generate a current state model representing a current state of the physical condition of the subject from body motion data of the subject over the current time period. Mathematical model 121 is constructed using the body motion data of the subject over a past period during which the physical condition of the subject was in a normal state,

Moreover, processor 12 uses mathematical model 121 to generate a normal state model representing the normal state of the physical condition of the subject from the body motion data of the subject over the past period. If there is no difference between the normal state model and the current state model, processor 12 may regenerate a normal state model representing the normal state of the subject from the body motion data of the subject over a new past period including the current time period. After that, the physical condition of the subject is to be detected using the regenerated normal state model.

Here, mathematical model 121 is constructed for each subject by evaluating a model generated (may also be referred to as trained) by constructing a neural network from a data set of the normal state, i.e., the body motion data of the subject over a past period during which the subject was in the normal state. In the present embodiment, mathematical model 121 is constructed using features obtained by calculating statistics that are based on interaction, from the body motion data of the subject over the past period. Note that, for example, the features include one or more of the following: a moving average (speed) of each of the heart rate components and the respiratory components, moving skewness (skewness of distribution) of each of the heart rate components and the respiratory components, dispersion (irregularity) of the respiratory components, and a moving outlier of the heart rate components (whether there is an abrupt change).

### [2.3 Outputter 13]

Outputter 13 outputs the state model generated by processor 12. More specifically, outputter 13 outputs the normal state model generated by processor 12, Moreover, outputter 13 outputs the current state model generated by processor 12 for detecting a change in the physical condition of the subject based on one or more differences between the normal state model and the current state model.

### [2.4 Operation etc. of Physical Condition Detection Device 10]

Next, operation etc. of physical condition detection device 10 configured as described above will be described.

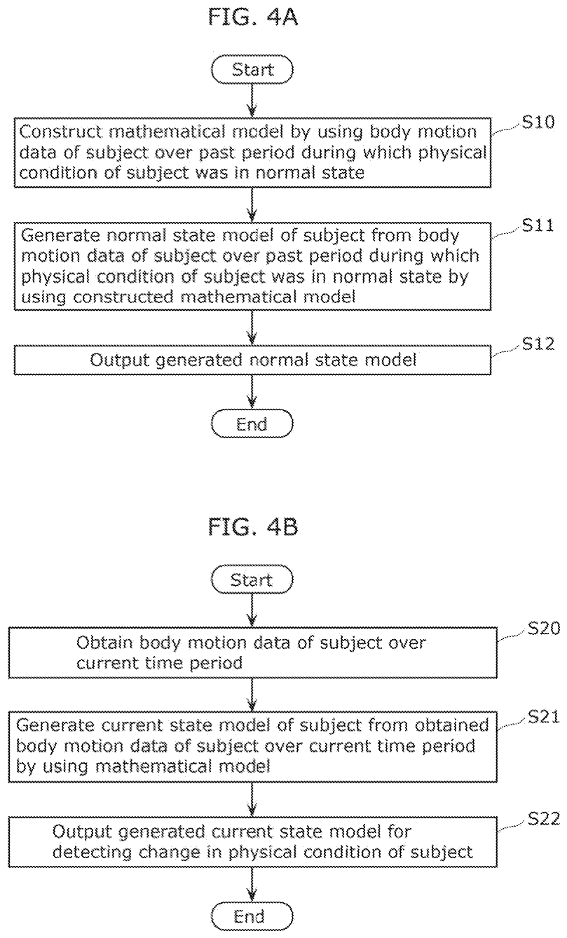

FIG. 4A and FIG. 4B are each a flowchart illustrating operation of physical condition detection device 10 according to the present embodiment. FIG. 4A illustrates operation starting from constructing a mathematical model to generating a normal state mode by physical condition detection device 10. FIG. 4B illustrates operation of generating a current state model by physical condition detection device 10.

As illustrated in FIG. 4A, mathematical model 121 is constructed first by using body motion data of a subject over a past period during which the subject was in a normal state (S10). In the present embodiment, mathematical model 121 is constructed using features obtained by calculating statistics that are based on interaction, from the body motion data of the subject over a past period during which the physical condition of the subject was in a normal state.

Next, a computer of physical condition detection device 10 generates a normal state model of the subject from the body motion data of the subject over the past period during which the physical condition of the subject was in the normal state by using mathematical model 121 constructed in step S10 (S11).

Next, the computer of physical condition detection device 10 outputs the normal state model generated in step S11 (S12).

Moreover, as illustrated in FIG. 4B, first, the computer of physical condition detection device 10 obtains body motion data of the subject over a current time period that is a given time period including a current time (S20).

Next, the computer of physical condition detection device 10 generates a current state model of the subject from the body motion data of the subject over the current time period, which is obtained in step S20, by using mathematical model 121 constructed in advance (S21).

Next, the computer of physical condition detection device 10 outputs the current state model generated in step S12 for detecting a change in the physical condition of the subject (S22).

When one or more differences between the normal state model and the current state model can be detected by comparing the output normal state model and the current state model on a display, etc., a change in the physical condition of the subject can be detected.

The following Working Example 1 and Working Example 2 will describe that a change in physical condition can be detected by comparing the normal state model and the current state model,

### WORKING EXAMPLE 1

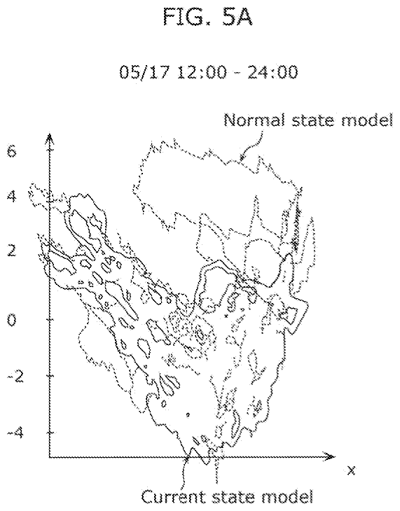

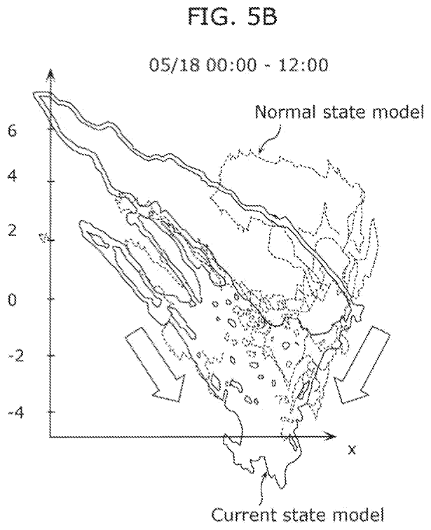

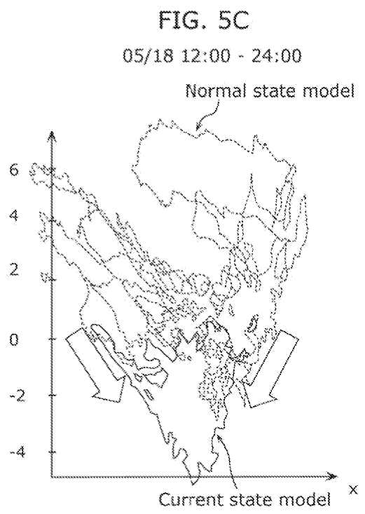

FIG. 5A through FIG. 5C are each a diagram illustrating a normal state model and a current state model according to Working Example 1 of the present embodiment.

In this working example, a subject whose change in the physical condition was to be detected was a patient of a hospice facility, and body motion data was obtained by room sensor 30 as illustrated in FIG. 3. In addition, the normal state model and the current state model illustrated in FIG. 5A through FIG. 5C were generated using body motion data obtained when subject 50 was sleeping in bed 60. FIG. 5A illustrates a current state model when the current time period was set to 12:00-24:00 on 5/17 (May 17). FIG. 5B illustrates a current state model when the current time period was set to 0:00-12:00 on 5/18 (May 18). FIG. 5C illustrates a current state model when the current time period was set to 12:00-24:00 on 5/18 (May 18). In addition, each of FIG. 5A through FIG. 5C also illustrates a normal state model over a past period when the physical condition of subject 50 was in a normal state. Note that this subject 50 died at around 24:00 on 5/20 (May 20).

As can be seen from FIG. 5A through FIG. 5C, the current state model gradually moved downward starting from 0:00-12:00 on 5/18 illustrated in FIG. 5B, i.e., starting from the morning of May 18, compared with the normal state model. Then, as illustrated in FIG. 5C, in 12:00-24:00 on 5/18, i.e, after noon on May 18, the current state model clearly moved downward, compared with the normal state model.

This shows that it is possible to detect a change in the physical condition of subject 50 on a daily basis such as a few days before, rather than on an hourly basis such as several hours before.

### WORKING EXAMPLE 2

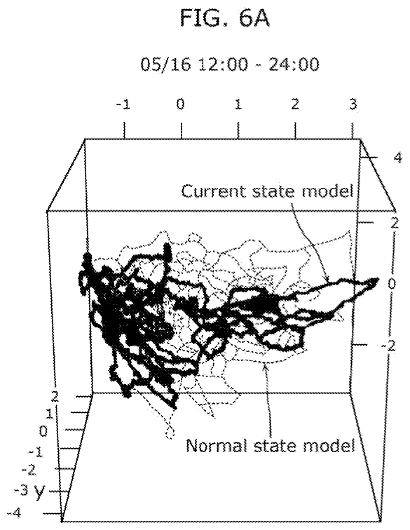

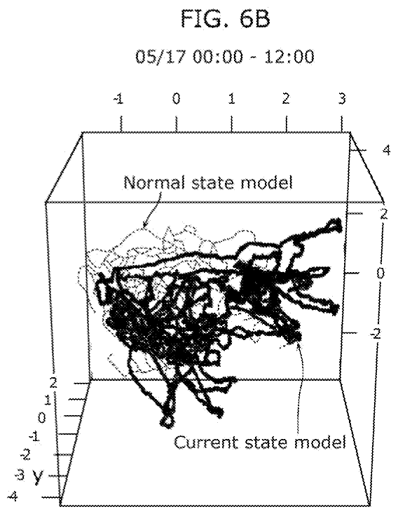

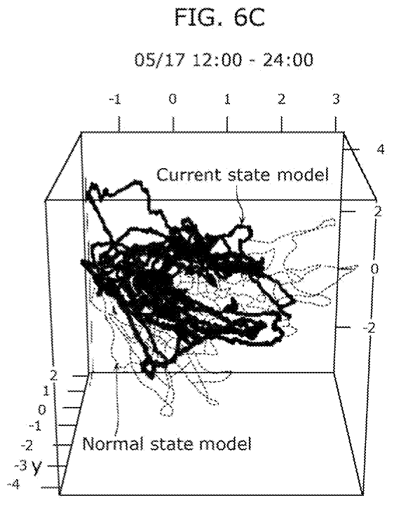

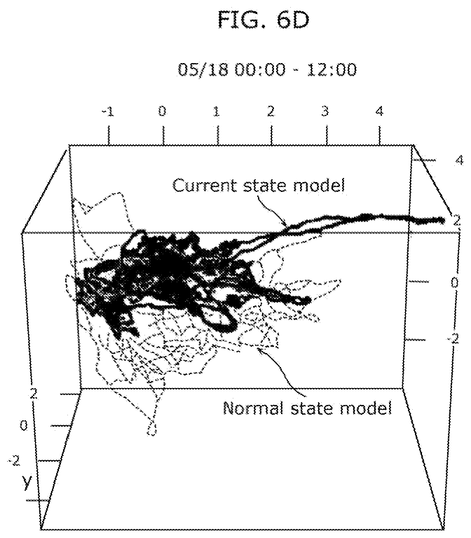

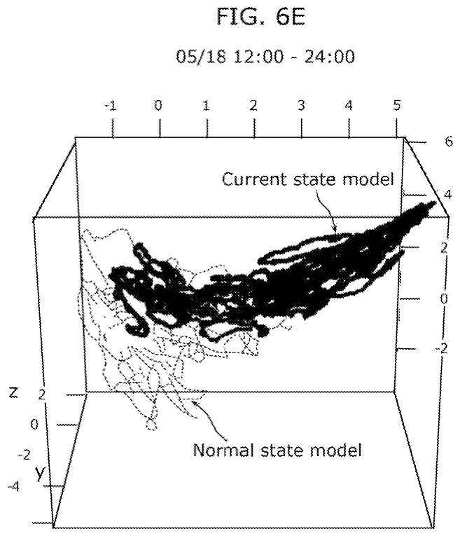

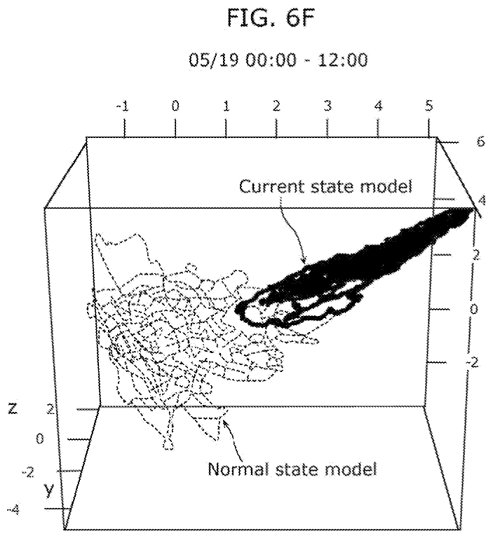

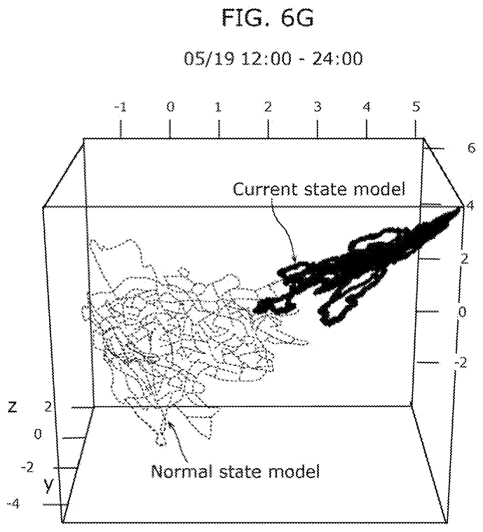

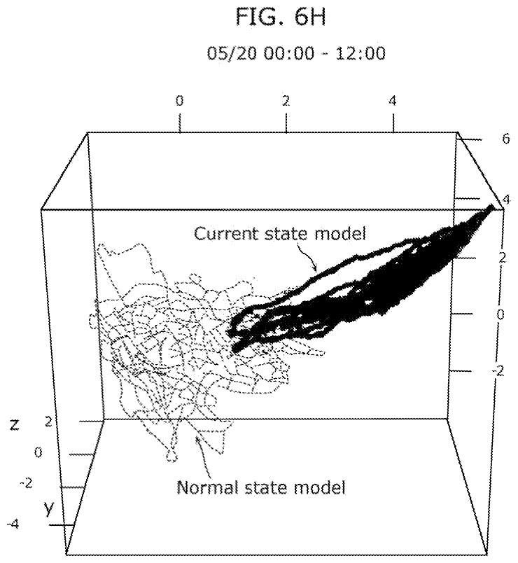

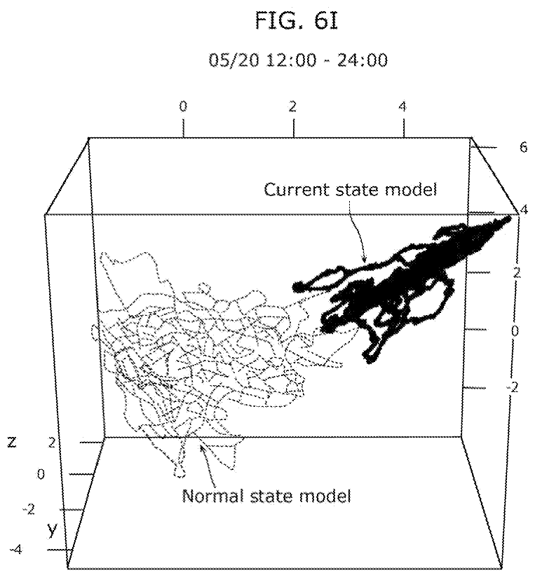

FIG. 6A through FIG. 6I are each a diagram illustrating a normal state model and a current state model according to Working Example 2 of the present embodiment.

In this working example, the subject is different from the subject in Working Example 1, but was a patient at a hospice facility. Body motion data of the subject was obtained by room sensor 30 as illustrated in FIG. 3. In addition, the normal state models and the current state models illustrated in FIG. 6A through FIG. 6I were generated using body motion data when subject 50 was sleeping in bed 60.

FIG. 6A illustrates a current state model when the current time was set to 12:00-24:00 on 5/16 (May 16). FIG. 6B illustrates a current state model when the current time period was set to 0:00-12:00 on 5/17 (May 17). FIG. 6C illustrates a current state model when the current time period was set to 12:00-24:00 on 5/17.

Moreover, FIG. 6D illustrates a current state model when the current time period was set to 0:00-12:00 on 5/18 (May 18). FIG. 6E illustrates a current state model when the current time period was set to 12:00-24:00 on 5/18 (May 18). FIG. 6F illustrates a current state model when the current time period was set to 0:00-12:00 on 5/19 (May 19). FIG. 6G illustrates a current state model when the current time period was set to 12:00-24:00 on 5/19 (May 19). FIG. 6H illustrates a current state model when the current time period was set to 0:00-12:00 on 5/20 (May 20). FIG. 6I illustrates a current state model when the current time period was set to 12:00-24:00 on 5/20 (May 20).

In addition, each of FIG. 6A through FIG. 6I also illustrates a normal state model over a past period when the physical condition of subject 50 was in a normal state. Note that this subject 50 died at around 24:00 on 5/20 (May 20).

As can be seen from FIG. 6A through FIG. 6H, the current state model deviated from the normal state model (outlier distribution) gradually from 0:00-12:00 on 5/18, i.e., the morning of May 18, illustrated in FIG. 6D. In addition, in 0:00-12:00 on 5/19 illustrated in FIG. 6F, i.e., in the morning of May 19, the current state model clearly moved to a different position, compared with the normal state model. Then, in 12:00-24;00 on 5/20 illustrated in FIG. 6I, i.e., after noon on May 20, the current state model moved to an entirely different position, compared with the normal state model.

Accordingly, the physical condition of subject 50 changed toward death two to three days before the death.

Therefore, if a relative of a patient who is subject 50 wishes to attend the deathbed of subject 50, a staff member of the hospice facility can detect a change in the physical condition of the patient on a daily basis, such as a few days before, and notify the relative. Therefore, the relative can have a time to rush to the patient and attend the patient's deathbed,

Note that physical condition detection device 10 may cause physical condition change detection device 20 to compare the normal state model and the current state model that are output by physical condition detection device 10 to detect a change in the physical condition of the patient. In the following, physical condition change detection device 20 will be described,

### [3. Physical Condition Change Detection Device 20]

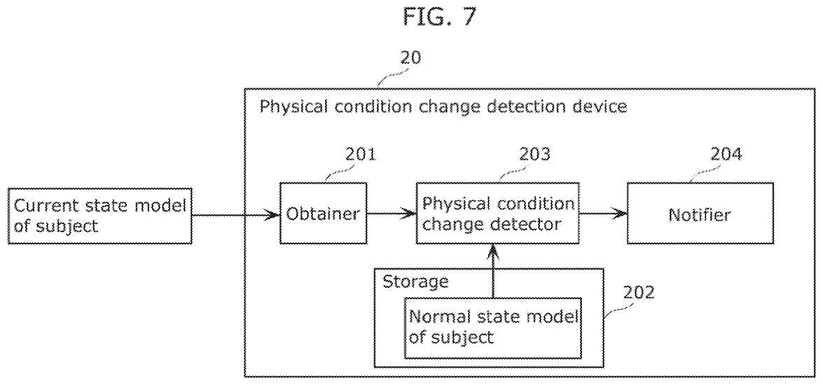

FIG. 7 is a diagram illustrating an example of a configuration of physical condition change detection device 20 according to the present embodiment.

Physical condition change detection device 20 can detect a change in physical condition of a subject by comparing the normal state model and the current state model that are output by physical condition detection device 10. In the present embodiment, as illustrated in FIG. 7, physical condition detection device 20 includes obtainer 201, storage 202, physical condition change detector 203, and notifier 204. Each structural element will be described in detail below.

### [3.1 Obtainer 201]

Obtainer 11 obtains the normal state model output by physical condition detection device 10 via network 40 in advance, and stores the normal state model in storage 202. Moreover, obtainer 11 obtains the current state model output by physical condition detection device 10 via network 40, Note that obtainer 11 obtains the current state model output by physical condition detection device 10 at each predetermined time.

### [3.2 Storage 202]

Storage 202 includes a non-volatile storage area and stores information to be used for various processing by physical condition change detection device 20. For example, storage 202 is read-only memory (ROM), flash memory, a hard disk drive (HDD), or the like. In the present embodiment, storage 202 stores the normal state model output by physical condition detection device 10. Moreover, storage 202 may temporarily store the current state nodel output by physical condition detection device 10.

### [3.3 Physical Condition Change Detector 203]

Physical condition change detector 203 compares the normal state model and the current state model to obtain one or more differences between the normal state model and the current state model, if there is any difference. Moreover, physical condition change detector 203 detects a change in the physical condition of the subject based on the one or more differences obtained by comparing the normal state model and the current state model.

Here, the one or more differences are (i) a distance between the normal state model and the current state model and (ii) a direction of movement from a position where the normal state model is located to a position where the current state model is located.

Here, for example, when description is given using the normal state model and the current state model illustrated in FIG. 6F, physical condition change detector 203 arranges the normal state model and the current state model in a space formed by the same coordinate axes, and compares, for example, the center of gravity of the distribution of the normal state model and the center of gravity of the distribution of the current state model. If physical condition change detector 203 detects any deviation between the center of gravity of the normal state model and the center of gravity of the current state model as a result of the comparison, physical condition change detector 203 may detect such deviation as one or more differences. Note that the center of gravity is an example of the distance between the normal state model and the current state model, and not limited to this example. Physical condition change detector 203 may arrange the normal state model and the current state model in a space formed by the same coordinate axes, and may compare whether there is a deviation between the portion where the normal state model is distributed in the space and the portion where the current state model is distributed in the space. In this case, physical condition change detector 203 can obtain, as one or more differences, (i) the distance between the portion where the normal state model is distributed in the space and the portion where the current state model is distributed in the space and (ii) the direction of movement from the portion where the normal state model is distributed in the space to the portion where the current state model is distributed in the space.

If there is no difference between the normal state model and the current state model, physical condition change detector 203 may notify physical condition detection device 10 via notifier 204 that there is no difference. This allows physical condition change detector 203 to trigger physical condition detection device 10 to regenerate the normal state model. Therefore, physical condition change detector 203 can update the normal state model stored in storage 202 to a fresh normal state model. Moreover, physical condition change detector 203 can detect a change in the physical condition of the subject more accurately by using such a fresh normal state model. In other words, it is possible to use a time period that is closer to a current time and during which the physical condition of the subject was in a normal state to generate the normal state model. Therefore, physical condition detection device 10 can detect a change in the physical condition of the subject more accurately.

### [3.4 Notifier 204]

Notifier 204 notifies an alert based on the one or more differences obtained by physical condition change detector 203. More specifically, notifier 204 notifies an alert when physical condition change detector 203 detects a change in the physical condition of the subject based on the obtained one or more differences. This makes it possible to know that there is a change in the physical condition of the subject or a change in the physical condition of the subject has been detected, upon receipt of the alert.

Note that notifier 204 may notify an alert on a mobile terminal, such as a smartphone, connected via network 40. With this, when (i) the subject whose change in the physical condition is to be detected is a patient at a hospice facility, (ii) a relative of subject 50 wishes to attend the deathbed of subject 50, and (iii) the physical condition of the subject changes toward death, an alert is notified on the mobile terminal of the relative or a staff member of the hospice facility. Therefore, the relative of the subject can know, the change in the physical condition of the subject directly or through the staff member of the hospice facility, on a daily basis such as a few days before. Therefore, the relative can have a time to rush to the subject and attend the deathbed of the subject.

### [4. Effects, etc.]

As described above, physical condition detection device 10 according to the present embodiment can generate and output a normal state model representing a normal state of the physical condition of the subject by using a mathematical model constructed from body motion data when the physical condition of the subject was in the normal state. Moreover, physical condition detection device 10 according to the present embodiment can generate and output a current state model representing a current state of the physical condition of the subject, from the body motion data of the subject in a current time period by using the constructed mathematical model. By comparing the normal state model and the current state model that are output by physical condition detection device 10, a change in the physical condition of the subject can be detected.

As described above, physical condition detection device 10 according to the present embodiment can output a current state model for detecting a change in the physical condition of the subject, and therefore a change in the physical condition of the subject can be detected.

### [Verification of Mathematical Model]

In the above embodiment, it has been described that mathematical model 121 is constructed for each subject using features obtained by calculating statistics that are based on interaction, from a data set in a normal state, that is, the body motion data of the subject in a past period during which the subject was in a normal state.

The following describes verification of the performance of the mathematical model constructed using features obtained by calculating various statistics, rather than the data set in a normal state as it is.

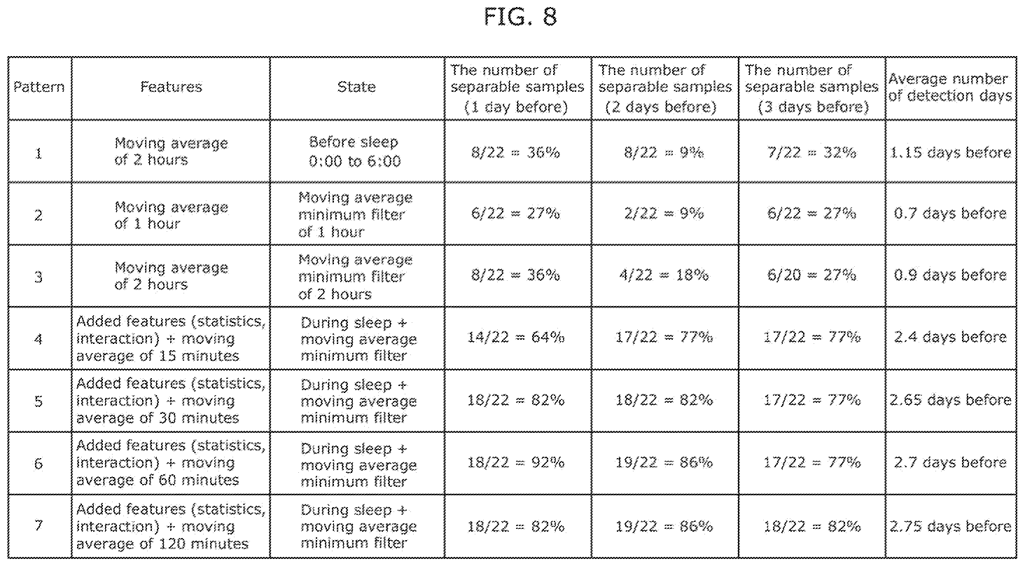

FIG. 8 is a table showing a result of verification of performance when a mathematical model was constructed using features of seven patterns. The body motion data used to construct the mathematical model were time-series body motion data, time-series heart rate data, and time-series respiration data in a normal state of each sampled subject during sleep. Moreover, the sampled subjects were 22 patients at a hospice facility, The mathematical models of 22 people were constructed using each feature of seven patterns. It is determined whether there is an overlap between (i) a region occupied by the mathematical model of each individual that was constructed using the features of each pattern and (ii) a region in which the body motion data of each individual is plotted on a daily basis one to three days before the date of death. The number of deviations that can be determined is used as the number of separable samples,

As shown in FIG. 8, in each of patterns 1 to 3, only features obtained by calculating a moving average from the body motion data were used to reduce variations in the time-series data. Patterns 1 to 3 differ only in time of the moving average. In contrast, in each of patterns 4 to 7, features were used that were obtained by calculating, from the same body motion data, (i) statistics that are based on interaction and (ii) a moving average, Note that, the statistics may be any values obtained by division or subtraction. Examples of the statistics include, but are not limited to, proportions of and one or more differences between time-series body motion data, time-series heart rate data, and time-series respiration data included in the body motion data. Patterns 4 to 7 differ from each other only in time of the moving average,

As can be seen from FIG. 8, the best average number of detection days was 2.75 days before. In addition, when the features obtained by calculating statistics that are based on interaction were used, such as in patterns 4 to 7, it was found that detection was possible at least 2.4 days before. In other words, constructing a mathematical model using features obtained by calculating statistics that are based on interaction makes it possible to detect a change in the body motion data of the subject, i.e., a change in the physical condition of the subject, two days before the date of death.

Physical condition detection device 10, etc. according to one or more aspects of the present disclosure have been described above on the basis of the embodiment and variations thereof, but the present disclosure is not limited to the embodiment and variations. One or more aspects of the present disclosure may include variations achieved by making various modifications to the present disclosure that can be conceived by those skilled in the art or forms achieved by combining structural components in different embodiments, without departing from the essence of the present disclosure. For example, the present disclosure also includes the following cases:

(1) Physical condition detection device 10 and physical condition change detection device 20 that have been described above may be used for detecting a change in physical condition due to pathology, including signs of myocardial infarction and the onset of apnea syndrome. In this case, the body motion data may include biological information that is necessary to detect a change in physical condition, such as weight information, as appropriate.

(2) Part of or all of the structural elements included in each of physical condition detection device 10 and physical condition change detection device 20 described above may include a computer system including a microprocessor, ROM, random-access memory (RAM), a hard disk unit, a display unit, a keyboard, a mouse, and so forth. The RAM or the hard disk unit stores a computer program. The microprocessor operating in accordance with the computer program enables each device to achieve its function, Here, the computer program is a combination of command codes that indicate instructions to the computer for achieving a predetermined function.

(3) Part of or all of the structural elements included in each of physical condition detection device 10 and physical condition change detection device 20 described above may include a single system large scale integration (LSI). A system LSI is a super-multifunctional LSI fabricated by integrating a plurality of structural elements on a single chip. The system LSI is more specifically a computer system that includes a microprocessor, ROM, RAM, and so forth. The RAM stores a computer program. The microprocessor operating in accordance with the computer program enables the system LSI to achieve its function.

(4) Part of or all of the structural elements included in each of physical condition detection device 10 and physical condition change detection device 20 described above may be implemented as an integrated circuit (IC) card or a single module removable from each of the devices. The IC card or the module is a computer system including a microprocessor, ROM, RAM, and so forth. The IC card or the module may include the super-multifunctional LSI described above. The microprocessor operating in accordance with the computer program enables the IC card or the module to achieve its function. This IC card or module may be tamper resistant,

### INDUSTRIAL APPLICABILITY

The present disclosure is applicable to a physical condition detection method, a physical condition detection device, and a recording medium to be used for detecting not only a change in physical condition toward death during terminal care, but also a change in physical condition due to pathology, including signs of myocardial infarction and the onset of apnea syndrome,

1. A physical condition detection method executed by a computer, the physical condition detection method comprising:
obtaining body motion data of a subject over a current time period that is a given time period including a current time;
generating a current state model representing a current state of physical condition of the subject from the body motion data of the subject over the current time period, by using a mathematical model constructed using body motion data of the subject over a past period during which the physical condition of the subject was in a normal state; and
outputting the current state model for detecting a change in the physical condition of the subject based on one or more differences obtained by comparing the current state model and a normal state model representing the normal state of the physical condition of the subject, the normal state model being generated from the body motion data of the subject over the past period using the mathematical model.

2. The physical condition detection method according to claim 1, further comprising:
obtaining the one or more differences by comparing the current state model and the normal state model; and
notifying an alert based on the one or more differences.

3. The physical condition detection method according to claim 2, wherein
the alert is notified when a change in the physical condition of the subject is detected based on the one or more differences.

4. The physical condition detection method according to claim 3, wherein
the one or more differences are (i) a distance between the normal state model and the current state model and (ii) a direction of movement from a position where the normal state model is located to a position where the current state model is located, and
the change in the physical condition of the subject is detected based on the distance and the direction.

5. The physical condition detection method according to claim 1, wherein
before the generating of the current state model, generating the normal state model from the body motion data of the subject over the past period by using the mathematical model.

6. The physical condition detection method according to claim 1, further comprising:
regenerating a normal state model representing the normal state of the subject from body motion data of the subject over a new past period including the current time period, when there is no difference between the normal state model and the current state model.

7. The physical condition detection method according to claim 1, wherein
the mathematical model is constructed using features obtained by calculating statistics that are based on interaction, from the body motion data of the subject over the past period.

8. A non-transitory computer-readable recording medium having a computer program recorded thereon for causing a computer to execute:
obtaining body motion data of a subject over a current time period that is a given time period including a current time;
generating a current state model representing a current state of physical condition of the subject from the body motion data of the subject over the current time period, by using a mathematical model constructed using body motion data of the subject over a past period during which the physical condition of the subject was in a normal state; and
outputting the current state model for detecting a change in the physical condition of the subject based on one or more differences obtained by comparing the current state model and a normal state model representing the normal state of the physical condition of the subject, the normal state model being generated from the body motion data of the subject over the past period using the mathematical model.

9. A physical condition detection device comprising:
an obtainer that obtains body motion data of a subject over a current time period that is a given time period including a current time;
a processor that generates a current state model representing a current state of physical condition of the subject from the body motion data of the subject over the current time period, by using a mathematical model constructed using body motion data of the subject over a past period during which the physical condition of the subject was in a normal state; and
an outputter that outputs the current state model for detecting a change in the physical condition of the subject based on one or more differences obtained by comparing the current state model and a normal state model representing the normal state of the physical condition of the subject, the normal state model being generated from the body motion data of the subject over the past period using the mathematical model.
